(19) United States
(12) Patent Application Publication
(10) Pub. No.: US 2018/0132939 A1
(43) Pub. Date: May 17, 2018

(54) HANDHELD 3D SCANNING DEVICE AND THE METHOD THEREOF

(71) Applicant: INDUSTRIAL TECHNOLOGY RESEARCH INSTITUTE, Hsinchu (TW)

(72) Inventors: Tung-Fa LIOU, Hsinchu City (TW); Po-Fu YEN, Taipei City (TW); Wen-Shiou LUO, Hsinchu City (TW); Chia-Chen CHEN, Hsinchu City (TW); Kang-Chou LIN, Nantou County (TW)

(73) Assignee: INDUSTRIAL TECHNOLOGY RESEARCH INSTITUTE, Hsinchu (TW)

(21) Appl. No.: 15/590,937

(22) Filed: May 9, 2017

(30) Foreign Application Priority Data
Nov. 16, 2016 (CN) 201611032660.3

(51) Int. Cl. A61B 34/10; H04N 13/02; G06F 1/16

(52) U.S. Cl. CPC A61B 34/10; A61B 2034/108; G06F 1/1633; H04N 13/0242

(57) ABSTRACT

In an embodiment of the disclosure, a handheld 3D scanning device is provided. The handheld 3D scanning device comprises at least one first 3D sensing module, at least one second 3D sensing module, and a fixing unit. Each of the at least one first 3D sensing module and the at least one second 3D sensing module comprises at least one projecting unit and at least one image sensing unit for performing a 3D scanning to an object to be measured. The fixing unit is provided to fix the at least one first 3D sensing module and the at least one second 3D sensing module at specific locations, respectively.

### CROSS-REFERENCE TO RELATED APPLICATION

This application claims the priority benefits of China application serial no. 201611032660.3, filed on Nov. 16, 2016. The entirety of each of the above-mentioned patent applications is hereby incorporated by reference herein.

### TECHNICAL FIELD

The disclosure relates to a handheld 3D scanning device and the method thereof.

### BACKGROUND

When the degree of physical injury is large, it often needs assistive devices in the process of treatment or rehabilitation. A three dimensional (3D) scanning may provide more accurate 3D shape information for manufacturing assistive devices, so as to manufacture the assistive device suitable for patient wearing.

At present, a single scanning range of the common handheld 3D scanner is limited, thereby it needs scanning the limb surround back and forth to obtain complete 3D information, which makes the scanning time longer and the operation is not convenient.

Besides, the normal human body scanner or foot scanner usually scan by a plurality of detectors hanged on a fixed track, which makes the volume of the scanner is huge, and needs the person to be measured to fix at a specific position and a specific posture. In this condition, some patients could not conduct the scanning operation with the machine. Therefore, how to make a 3D scanning device that scan the upper limbs or the lower limbs of patients rapidly and accommodate the physical condition of patients at the same time for being adapted to a variety of body postures is an issue needed to overcome now.

### SUMMARY

The embodiments of the disclosure provide a handheld 3D scanning device, which may rapidly scan the upper limbs or the lower limbs of patients, and it may scan depending on the limb conditions of patients and any limb posture, for reducing the difficulty of scanning and the scanning time, and enhancing the convenience of use.

In an embodiment of the disclosure, a handheld 3D scanning device is provided. The handheld 3D scanning device comprises at least one first 3D sensing module, at least one second 3D sensing module, and a fixing unit. Each of the at least one first 3D sensing module and the at least one second 3D sensing module comprises at least one projecting unit and at least one image sensing unit for performing a 3D scanning to an object to be measured. The fixing unit is provided to fix the at least one first 3D sensing module and the at least one second 3D sensing module at specific locations, respectively.

In another embodiment of the disclosure, a handheld 3D scanning method adapted to a handheld 3D scanning device is provided. It comprises: (a) obtaining a plurality of 3D measuring information transfer matrices respectively corresponding to at least one first 3D sensing module and at least one second 3D sensing module by a calibration method; (b) obtaining a plurality of 3D measuring information of the object to be measured by the at least one first 3D sensing module and the at least one second 3D sensing module; (c) integrating the plurality of 3D measuring information according to the corresponding 3D measuring information transfer matrices into a first position 3D data; (d) moving the handheld 3D scanning device to a second position and obtaining a second position 3D data; (e) comparing the second position 3D data and the first position 3D data and timely integrating them to obtain an integrating 3D data; (f) moving the handheld 3D scanning device to a next position to obtain a next position 3D data, then comparing the next position 3D data and the integrating 3D data of a previous position and timely integrating them for obtaining a new integrating 3D data for the next position; and (g) repeating the step (f) until completing a whole scanning to the object and obtaining a completely integrating 3D data.

The foregoing will become better understood from a careful reading of a detailed description provided herein below with appropriate reference to the accompanying drawings.

### BRIEF DESCRIPTION OF THE DRAWINGS

FIG. 1a is a schematic diagram of a handheld 3D scanning device according to an embodiment of the disclosure.

FIG. 1b is a schematic diagram of a dismantled handheld 3D scanning device according to FIG. 1a.

FIG. 2a is a schematic diagram of a handheld 3D scanning device according to an embodiment of the disclosure.

FIG. 2b is a schematic diagram of a dismantled handheld 3D scanning device according to FIG. 2a.

FIG. 3a is a schematic diagram of a handheld 3D scanning device, wherein the fixing unit has an opening, according to another embodiment of the disclosure.

FIG. 3b is a schematic diagram of a handheld 3D scanning device, wherein the fixing unit has an opening, according to the other embodiment of the disclosure.

FIG. 4a and FIG. 4b are schematic diagrams of a holding part of the handheld 3D scanning device according to two embodiments of the disclosure, respectively.

FIG. 5a, FIG. 5b and FIG. 5c are configuration diagrams of the 3D sensing module according to an embodiment of the disclosure respectively.

FIG. 6 is an operating flow of a handheld 3D scanning method adapted to the handheld 3D scanning device according to an embodiment of the disclosure.

FIG. 7 is an operating flow of a calibration method of the handheld 3D scanning device according to an embodiment of the disclosure.

### DESCRIPTION OF THE EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

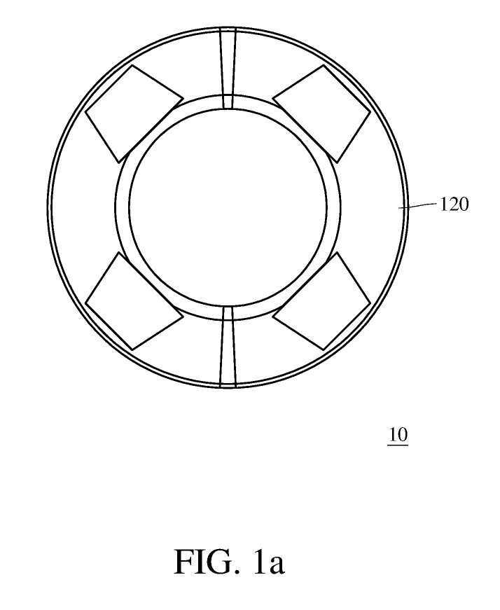

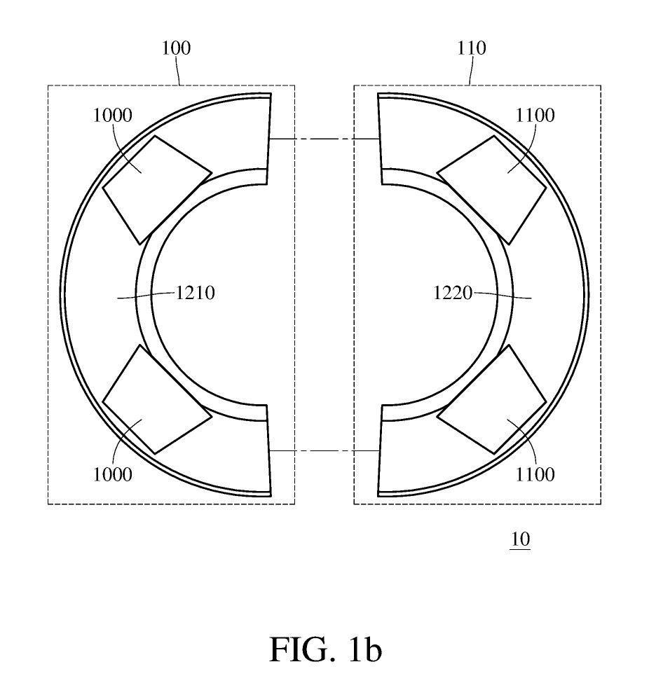

FIG. 1a is a schematic diagram of a handheld 3D scanning device according to an embodiment of the disclosure, and FIG. 1b is a schematic diagram of a dismantled handheld 3D scanning device according to FIG. 1a. Please refer to FIG. 1a and FIG. 1b, a handheld 3D scanning device 10 comprises at least one first 3D sensing module 1000, at least one second 3D sensing module 1100 and a fixing unit 120. Each of the at least one first 3D sensing module 1000 and the at least one second 3D sensing module 1100 comprises a projecting unit and an image sensing unit (not shown in the figures), so as to project to an object to be measured and capture an image for 3D measurement. In this embodiment, each of the amount of the at least one first 3D sensing module 1000 and the amount of the at least one second 3D sensing module 1100 is two, but the embodiment does not limit the scope of the disclosure. The amount of the at least one first or second 3D sensing module may increase or decrease according to the demand.

The fixing unit 120 is provided to fix the at least one first 3D sensing module 1000 and the at least one second 3D sensing module 1100 to specific positions, respectively, so as to insure a relative position relationship between 3D sensing modules, and the relative position relationship may be used as a parameter for the following 3D information calculation. The fixing unit 120 comprises a first fixing part 1210 and a second fixing part 1220, and the fixing part and the second fixing part are symmetrical with each other on a plane. The connecting way of the first fixing part 1210 and the second fixing part 1220 may be a positioning mechanism, such as location pins or switch fasteners, or a magnetic suction, so as to split the handheld 3D scanning device 10 into a first portion 100 and a second portion 110. Therefore, the handheld 3d scanning device 10 may be split rapidly and put the limbs into a detecting area and then connect the fixing part for executing a 3D scanning according to the physical condition of limbs. The first fixing part and the second fixing part are symmetrical with each other on a plane in this embodiment, but the embodiment does not limit the scope of the disclosure. The first fixing part and the second fixing part may not be on a plane or may be asymmetrical, as long as they form a ring, a polygon or the like structure. Besides, the fixing unit 120 may be split into two fixing parts in this embodiment, but the embodiment does not limit the scope of the disclosure. The fixing unit may be split into more fixing parts according to the demand, and each dismantled fixing part and the 3D sensing module disposed thereon may perform a 3D scanning measurement independently.

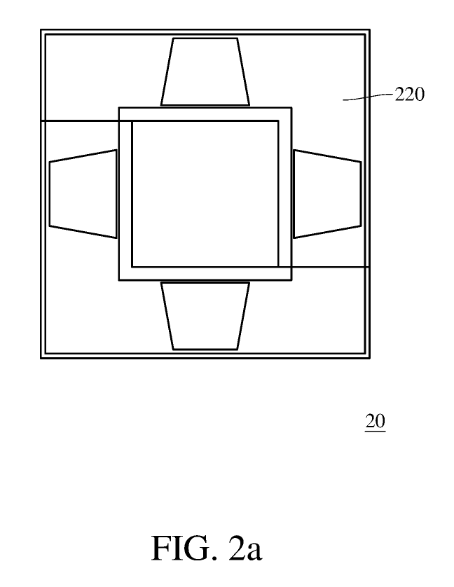

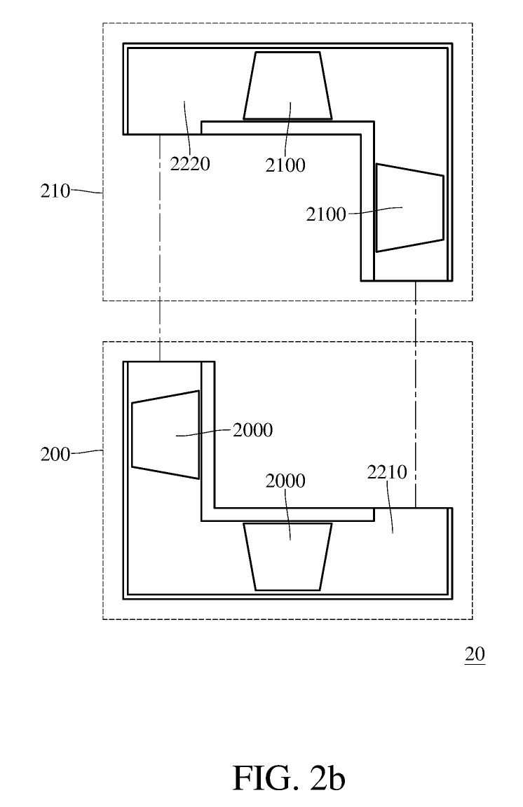

FIG. 2a is a schematic diagram of a handheld 3D scanning device according to an embodiment of the disclosure, and FIG. 2b is a schematic diagram of a dismantled handheld 3D scanning device according to FIG. 2a. The fixing unit may be divided into a first part 200 and a second part 210. A plurality of first 3D sensing module 2000 are fixed to the first fixing part 2210, and a plurality of second 3D sensing module 2100 are fixed to the second fixing part 2220. In the embodiment according to FIG. 1a and FIG. 1b, the fixing unit 120 is a circular ring, and each of the first fixing part 1210 and the second fixing part 1220 are a semi-circular ring. In the embodiment according to FIG. 2a and FIG. 2b, the fixing unit 220 is a rectangular ring, and each of the first fixing part 2210 and the second fixing part 2220 are L-shaped. But the shape of the fixing unit is not limited thereto, a ring structure of the shape may be an ellipse, a parabolic shape, a polygon or any irregular-shaped ring according to the demand.

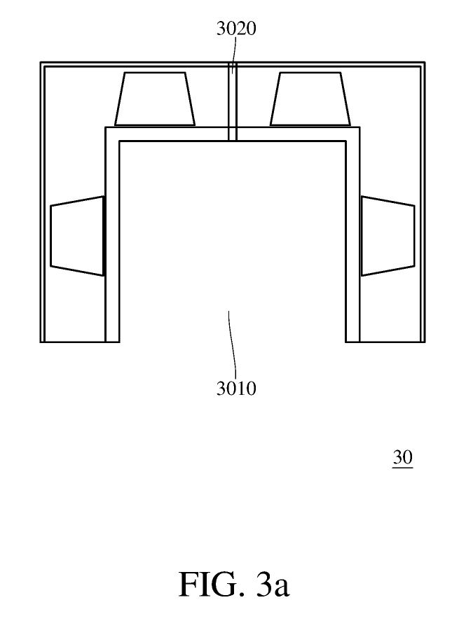

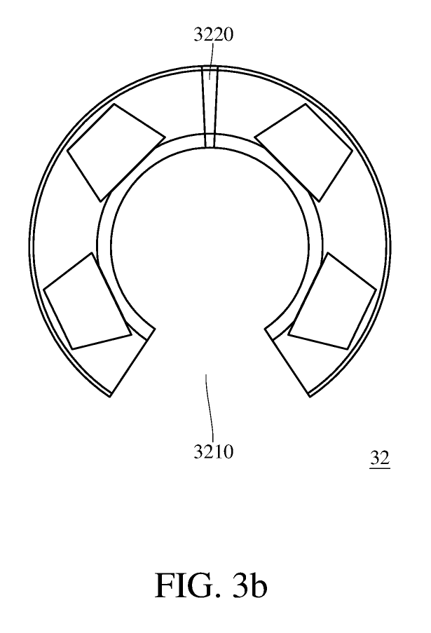

Besides, in order to facilitate the placement or the removal of limbs, the fixing unit may have an opening, as shown in the embodiments of FIG. 3a and FIG. 3b, respectively. The handheld 3D scanning device 30 comprises an opening 3010, and the first fixing part and the second fixing part are connected through a connecting part 3020. The handheld 3D scanning device 32 comprises an opening 3210, and the first fixing part and the second fixing part are connected through a connecting part 3220. In addition, the size range of the opening may also design according to the demand. For example, the handheld 3D scanning device 30 is fl shaped, and the size range of the opening is narrowed, to have the fourth side of the handheld 3D scanning device 30 still having partial fixing unit, but the aforesaid embodiments do not limit the scope of the disclosure.

Moreover, the fixing unit according to the aforesaid embodiments may be dismantled into the first fixing part and the second fixing part, but the aforesaid embodiments do not limit the scope of the disclosure. The fixing unit may be dismantled into three fixing parts, four fixing part or more fixing parts. Or the number of fixing parts that are dismantled may be decided according the number of 3D sensing modules, which may be adjusted according to the demand.

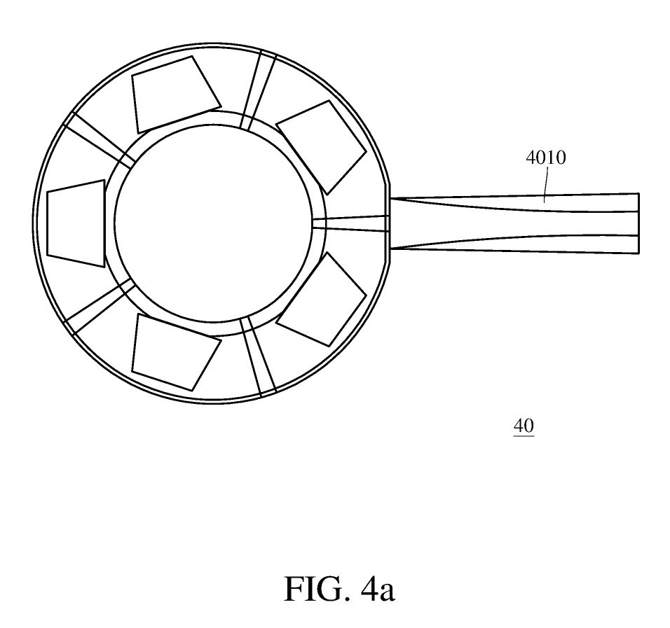

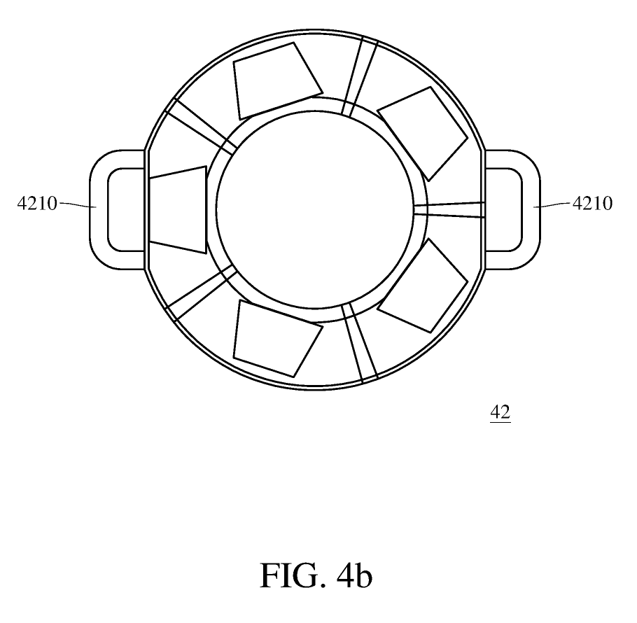

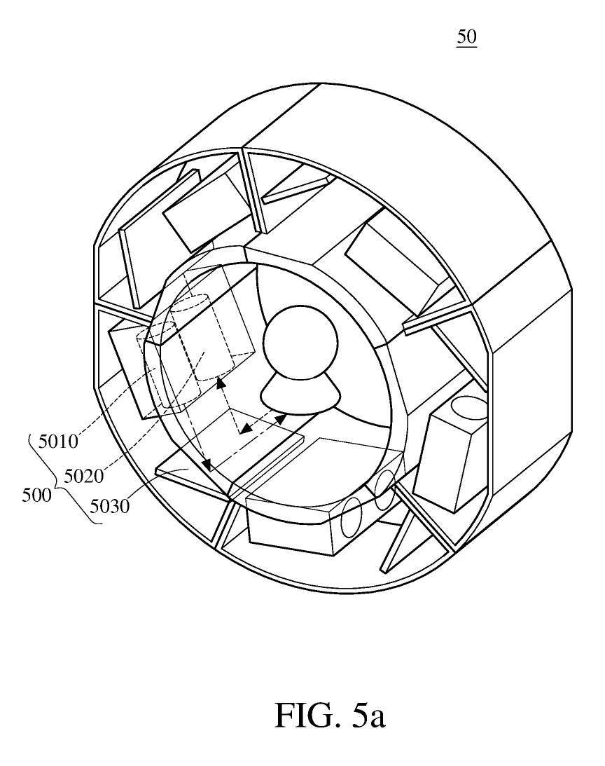

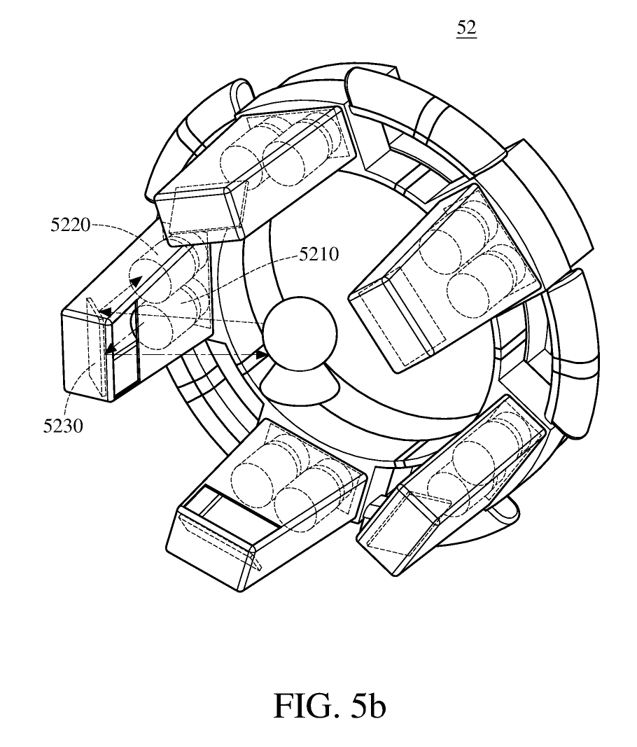

In order to facilitate the handheld scanning, the handheld 3D scanning device may comprise a handheld part, as shown in two embodiments of FIG. 4a and FIG. 4b, respectively. The handheld 3D scanning device 40 comprises a handheld part 4010 for facilitating handheld scanning. Besides, the handheld parts 4210 of the handheld 3D scanning device 42 may be disposed on two sides of the device, respectively, for facilitating handheld scanning with two hands. But the embodiment does not limit the scope of the disclosure, and the position and the shape of the handheld part may be changed according to the demand.

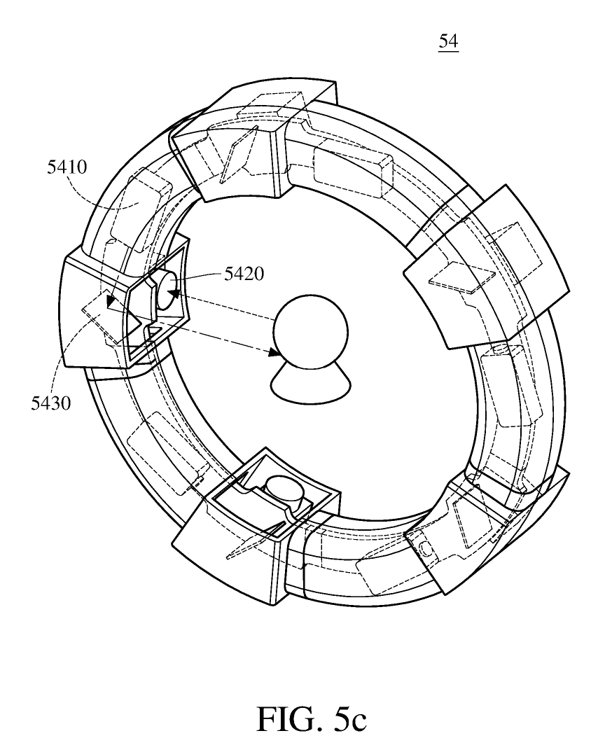

FIG. 5a, FIG. 5b and FIG. 5c are configuration diagrams of the 3D sensing module according to an embodiment of the disclosure respectively. As shown in FIG. 5a, a 3D sensing module 500 of a handheld 3D scanning device 50 comprises a projecting unit 5010, an image sensing unit 5020 and a reflector 5030. The projecting unit 5010 projects an image along a tangential direction of the ring and the image is projected onto an object to be measured and further reflected by the reflector 5030. And the image reflected from the object to be measured is reflected by the reflector 5030 and further projected onto the image sensing unit 5020. The image sensing unit 5020 receives the image, and then performs a 3D measuring calculation according to the received image.

Please refer to FIG. 5b, the projecting unit 5210 projects an image along the radial direction of the ring, and the image is projected onto the object to be measured and further reflected by the reflector 5230. The image reflected from the object to be measured is further projected onto an image sensing unit 5220 by the reflector 5230, and then the image sensing unit 5220 performs the 3D measuring calculation according to the received image. The difference between FIG. 5b and FIG. 5a is the projecting direction of the 3D sensing module in FIG. 5b is towards a radial direction. For the embodiment of FIG. 5b, the occupied area of the 3D sensing module in the 3D scanning device becomes smaller, which makes the handheld 3D scanning device to accommodate more 3D sensing modules. Besides, the 3D sensing modules in FIG. 5b may be separated from the fixing unit individually, which enhances the convenience of use.

Sometimes, for saving the space, the reflector is independently disposed on the light path of projecting or image sensing. As shown in FIG. 5c, different from FIG. 5a and FIG. 5b, the reflector shown in FIG. 5c is only used for projecting unit. The projecting unit 5410 projects an image along the tangential direction of the ring and the image is projected by the reflector 5430 onto the object to be measured. And the image reflected from the object to be measured is directly received by the image sensing unit 5420, and then the image sensing unit 5420 performs the 3D measuring calculation according the received image.

The handheld 3D scanning device of the disclosure comprises a plurality of 3D sensing modules. When each of these 3D sensing modules is working, it may use the time-sharing scanning or frequency dividing scanning to prevent mutual interference between these 3D sensing modules. For example, each 3D sensing module may perform time-sharing scanning (for example, perform scanning at different time points sequentially), or the light source wavelengths of the projecting units of these 3D sensing modules are different, so as to prevent interference when these 3D sensing modules capture images at the same time.

The handheld 3D scanning device of the disclosure needs not limit the scanning moving path strictly, and it may complete the 3D scanning. Besides, the handheld 3D scanning device of the disclosure may combine a track for electronically automatic scanning or manually scanning to enhance the convenience of use. Therefore, the handheld 3D scanning device may further comprises a track connecting device. The handheld 3D scanning device may connect the track through the track connecting device, and perform the 3D scanning along the track.

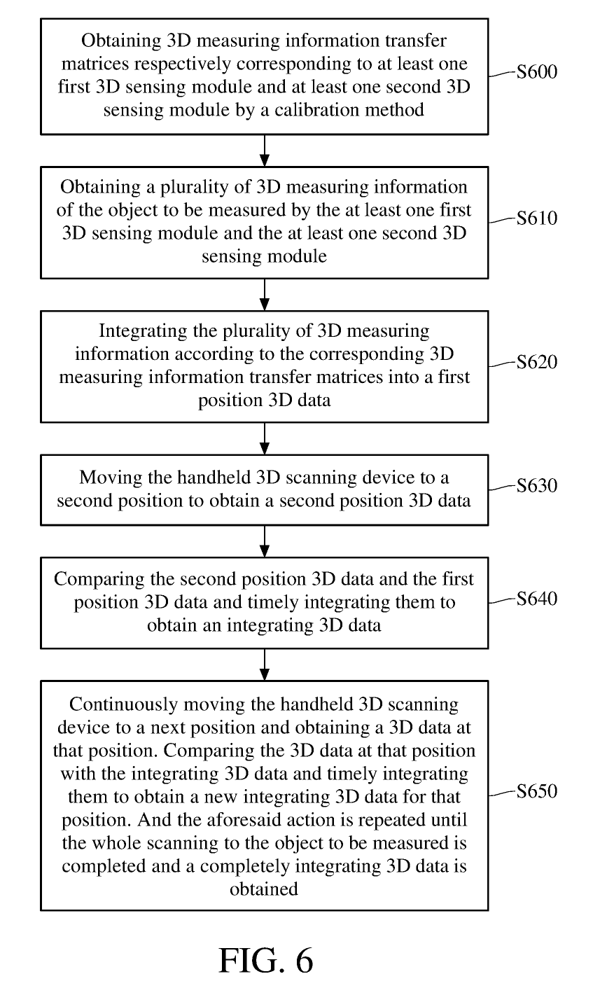

FIG. 6 is an operating flow of a handheld 3D scanning method adapted to the handheld 3D scanning device according to an embodiment of the disclosure. First, the step S600 may include obtaining a plurality of 3D measuring information transfer matrices respectively corresponding to at least one first 3D sensing module and at least one second 3D sensing module by a calibration method. The calibration method will be described in detail in the following paragraph. Next, the step S610 may include capturing image by the at least one first 3D sensing module and the at least one second 3D sensing module, and obtaining a plurality of 3D measuring information. The step S620 may include integrating the plurality of 3D measuring information according to the corresponding 3D measuring information transfer matrices into a first position 3D data. After that, step S630 may include moving the handheld 3D scanning device to a second position and capturing image by the at least one first 3D sensing module and the at least one second 3D sensing module, then obtaining a plurality of 3D measuring information, and integrating the plurality of 3D measuring information according the corresponding 3D measuring information transfer matrices into a second position 3D data. The step S640 may include comparing the second position 3D data and the first position 3D data and timely integrating them to obtain an integrating 3D data. The step S650 may include continuously moving the handheld 3D scanning device to a next position and obtaining a 3D data at that position, and comparing the 3D data at that position with the integrating 3D data and timely integrating them to obtain a new integrating 3D data for the next position. The aforesaid action (step 650) is repeated until the whole scanning to the object to be measured is completed and a completely integrating 3D data is obtained. Therefore, the handheld 3D scanning device of the disclosure needs not scan back and forth. Instead, the handheld 3D scanning device of the disclosure only needs to move one time then completes the 3D scanning, which means that the handheld 3D scanning device only needs to continuously move forward along one direction. And when the handheld 3D scanning device moves to a new added region, the 3D data is accumulated with the timely integration, thereby after finishing the scanning movement, the handheld 3D scanning device may obtain the complete 3D data of the object to be measured in real time.

Besides, the scope of the present disclosure is not limited to handheld scanning, it may combine with a track for scanning by a manually moving way or an electronically-controlled automatic moving way, but the scope of the disclosure is not limited thereto. Furthermore, for preventing mutually interference when each of 3D sensing modules is working, time-sharing scanning or frequency dividing scanning may be used.

When the time-sharing scanning is performed, such as in the step S610, each of the first 3D sensing modules and the second sensing modules may project and capture images to the object to be measured at different time points, and obtain a plurality of 3D measuring information. When the frequency dividing scanning is performed, each of the first 3D sensing modules and the second 3D sensing modules may adopt the projecting light source having different frequencies to project. And the image sensing unit may filter the light correspondingly and only receives the light having the corresponding frequency. Therefore, each 3D sensing module will not receive the light signal of other 3D sensing modules of these 3D sensing modules, so that the obtained 3D measuring information will not be mutually interfered. Besides, the frequency dividing scanning needs not let each 3D sensing module adopting the projecting light source having different frequencies. If the projecting areas between the 3D sensing modules do not overlap or interfere with each other, then the projecting light source with the same frequency may be adopted. The projecting light source having different frequencies and the correspondingly filtering image sensing unit are adopted only when the overlapping projecting or interference occurs. This may be adjusted according to the demand, and the scope of the disclosure is not limited thereto.

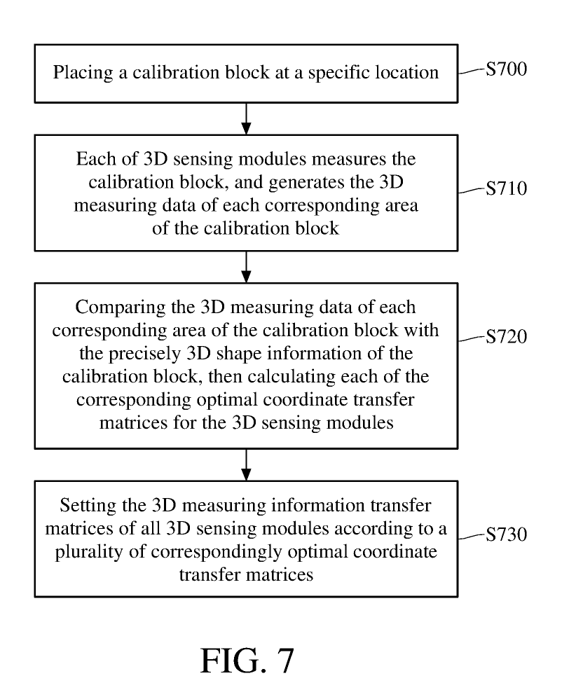

The handheld 3D scanning device of the disclosure may comprise two or more 3D sensing modules, therefore the factory calibration is required before the 3D scanning is performed. FIG. 7 is an operating flow of a calibration method of the handheld 3D scanning device according to an embodiment of the disclosure. As shown in FIG. 7, the step S700 may include placing a calibration block with a known shape at a specific location. The calibration block has an asymmetric shape, and the precisely 3D shape information (or called Computer-Aided Design (CAD) model) of the calibration block may be designed in advance and then is generated by a precision machining method to make a designed 3D shape into an entity block. Or it may freely choose an entity block with asymmetric shape, and measure and obtain the precisely 3D shape information of the calibration block by using accurate 3D measuring equipment. Or other methods which may obtain the precisely 3D shape information of the calibration block may be used, but the scope of the disclosure is not limited thereto.

In the step S710, each of 3D sensing modules measures the calibration block, and generates the 3D measuring data of each corresponding area of the calibration block. Then step S720 may include taking the precisely 3D shape information of the calibration block as a base, and comparing with the 3D measuring data of each corresponding area of the calibration block, then calculating each of the corresponding optimal coordinate transfer matrices for the 3D sensing modules, respectively. It should be noted that, the disposing location and the precisely 3D shape information of the calibration block define a world coordinate system, and each 3D measuring data of each 3D sensing module will be transferred to the common world coordinate system.

Assuming Pₙ⁽ⁱ⁾ is a nth measuring data of the ith sensing head, Qₙ⁽ⁱ⁾ is a corresponding point in the CAD Model. The Pₙ⁽ⁱ⁾ is transferred by a coordinate transfer matrix T⁽ⁱ⁾ from the sensing head coordinate to the CAD Model coordinate system. T⁽ⁱ⁾ is a 3*4 matrix, which is defined as following:

$${T}^{( i )} = [ \begin{matrix}{t}_{0}^{( i )} & {t}_{1}^{( i )} & {t}_{2}^{( i )} & {t}_{3}^{( i )} \\ {t}_{4}^{( i )} & {t}_{5}^{( i )} & {t}_{6}^{( i )} & {t}_{7}^{( i )} \\ {t}_{8}^{( i )} & {t}_{9}^{( i )} & {t}_{10}^{( i )} & {t}_{11}^{( i )}\end{matrix} ]$$

All the measuring data Pₙ⁽ⁱ⁾ are transferred to the nearest corresponding point Qₙ⁽ⁱ⁾ by the optimal coordinate transfer matrix T⁽ⁱ⁾, which makes the distance between the point group Pₙ⁽ⁱ⁾ and the point group Qₙ⁽ⁱ⁾ is minimized. And it may be described as below:

$$MIN 〈 {\sum }_{n}^{ } ‖ ( [ \begin{matrix}{t}_{0}^{( i )} & {t}_{1}^{( i )} & {t}_{2}^{( i )} \\ {t}_{4}^{( i )} & {t}_{5}^{( i )} & {t}_{6}^{( i )} \\ {t}_{8}^{( i )} & {t}_{9}^{( i )} & {t}_{10}^{( i )}\end{matrix} ] {P}_{n}^{( i )} + [ \begin{matrix}{t}_{3}^{( i )} \\ {t}_{7}^{( i )} \\ {t}_{11}^{( i )}\end{matrix} ] ) - {Q}_{n}^{( i )} ‖ 〉$$

It may use artificial adjustment methods first for rough alignment, and get the rough transfer matrix for transferring the measuring data Pₙ⁽ⁱ⁾ to the CAD model. If the angle difference and the deviation in the three coordinate axis which is perpendicular to each other between the measuring data and the CAD Model are roughly estimated, then a roughly transfer matrix may be calculated. After that, a method of iterative closest point (ICP) for automatically fine tune may be adopted. Finally, the measuring data Pₙ⁽ⁱ⁾ is precisely transferred to the CAD Model, and the optimal transfer matrix T⁽ⁱ⁾ is obtained.

At last, the step S730 may include setting the 3D measuring information transfer matrix of the 3D sensing module according to a plurality of correspondingly optimal coordinate transfer matrices, for example, setting each of the plurality of optimal coordinate transfer matrices to be a 3D measuring information transfer matrix of a corresponding 3D sensing module of the at least one first 3D sensing module and the at least one second 3D sensing module.

In summary, the handheld 3D scanning device according to the embodiments of the disclosure adopts a plurality of sets of 3D sensing modules placed towards a central area of the ring, and fixed at a specific position of a fixing unit for scanning the object to be measured. The fixing unit may be rapidly dismantled and assembled, and without extra calibration. Therefore, the handheld 3D scanning device according to the embodiments of the disclosure may perform scanning according to limb conditions of patients and a variety of limb postures, and only need to scan one time without back and forth scanning to complete the whole limb scanning. It reduces the difficulty of scanning and the scanning time, and enhances the convenience of use.

It will be apparent to those skilled in the art that various modifications and variations can be made to the disclosed embodiments. It is intended that the specification and examples be considered as exemplary embodiments only, with a scope of the disclosure being indicated by the following claims and their equivalents.

What is claimed is:

1. A handheld three dimensional (3D) scanning device, comprising:
at least one first 3D sensing module and at least one second 3D sensing module, wherein each of the at least one first 3D sensing module and the at least one second 3D sensing module comprises at least one projecting unit and at least one image sensing unit for performing a 3D scanning to an object to be measured; and
a fixing unit, provided to fix the at least one first 3D sensing module and the at least one second 3D sensing module at specific locations, respectively.

2. The handheld 3D scanning device according to claim 1, wherein the fixing unit comprises a first fixing part and a second fixing part, and the at least one first 3D sensing module is fixed to the first fixing part, the at least one second 3D sensing module is fixed to the second fixing part.

3. The handheld 3D scanning device according to claim 2, wherein a shape of the fixing unit is a ring structure.

4. The handheld 3D scanning device according to claim 3, wherein the ring structure is a circle, an ellipse, a parabolic shape, a polygon or an irregular-shaped ring.

5. The handheld 3D scanning device according to claim 2, wherein the fixing unit further comprises an opening, for facilitating a placement or a removal of the object to be measured.

6. The handheld 3D scanning device according to claim 2, wherein the fixing unit further comprises a handheld part, the handheld part is connected with the fixing unit for performing the 3D scanning by holding the handheld part.

7. The handheld 3D scanning device according to claim 2, wherein the first fixing part and the second fixing part are connected through a magnetic suction or a mechanism positioning structure, for dismantling the handheld 3D scanning device and placing the object to be measured, then connecting the handheld 3D scanning device for performing the 3D scanning.

8. The handheld 3D scanning device according to claim 1, further comprises a track connecting device, and the handheld 3D scanning device performs the 3D scanning along the track.

9. The handheld 3D scanning device according to claim 1, wherein light source wavelengths of the projecting units of the at least one first 3D sensing module and the at least one second 3D sensing module are different.

10. The handheld 3D scanning device according to claim 3, wherein the at least one first 3D sensing module and the at least one second 3D sensing module further comprise a reflector, to have a projecting light from the projecting unit reflecting along a tangential direction of a ring to a radial direction of the ring then projecting onto the object to be measured, and the projecting light along the radial direction of the ring is further reflected by the reflector from the object to the tangential direction, and then enters the at least one image sensing unit.

11. The handheld 3D scanning device according to claim 3, wherein those 3D sensing module further comprises a reflector, to have a projecting light from the projecting unit reflecting along a radial direction of a ring to a tangential direction of the ring then projecting onto the object to be measured, and the projecting light along the tangential direction of the ring is further reflected by the reflector from the object to the radial direction, and then enters the at least one image sensing unit.

12. The handheld 3D scanning device according to claim 1, wherein the at least one first 3D sensing module and the at least one second 3D sensing module further comprises a reflector, and the reflector is disposed on a light path of either the at least one projecting unit or the at least one image sensing unit.

13. A handheld three dimensional (3D) scanning method, adapted to the handheld 3D scanning device according to claim 1, comprising:
(a) obtaining a plurality of 3D measuring information transfer matrices respectively corresponding to the at least one first 3D sensing module and the at least one second 3D sensing module by a calibration method;
(b) obtaining a plurality of 3D measuring information of the object to be measured by the at least one first 3D sensing module and the at least one second 3D sensing module;
(c) integrating the plurality of 3D measuring information according to the corresponding 3D measuring information transfer matrices into a first position 3D data;
(d) moving the handheld 3D scanning device to a second position and obtaining a second position 3D data;
(e) comparing the second position 3D data and the first position 3D data and timely integrating them to obtain an integrating 3D data;
(f) moving the handheld 3D scanning device to a next position to obtain a next position 3D data, then comparing the next position 3D data and the integrating 3D data of a previous position and timely integrating them for obtaining a new integrating 3D data for the next position; and
(g) repeating the step (f) until completing a whole scanning to the object and obtaining a completely integrating 3D data.

14. The handheld 3D scanning method according to claim 13, wherein the calibration method comprises:
placing a calibration block at a specific location;
measuring the calibration block by the at least one first 3D sensing module and the at least one second 3D sensing module and generating a plurality of 3D measuring data of each corresponding area of the calibration block;
comparing the plurality of 3D measuring data and 3D shape information of the calibration block, and correspondingly calculating a plurality of optimal coordinate transfer matrices; and
setting each of the plurality of optimal coordinate transfer matrices to be a 3D measuring information transfer matrix of a corresponding 3D sensing module of the at least one first 3D sensing module and the at least one second 3D sensing module.

15. The handheld 3D scanning method according to claim 14, wherein the calibration block is asymmetric shaped.

16. The handheld 3D scanning method according to claim 15, wherein the calibration block is generated by a precision machining method to make a designed 3D shape into an entity block, and the 3D shape information of the calibration block is the information of the designed 3D shape.

17. The handheld 3D scanning method according to claim 15, wherein the calibration block is an entity block with the asymmetric shape, and the 3D shape information of the calibration block is obtained by using accurate 3D measuring equipment.

18. The handheld 3D scanning method according to claim 13, wherein the steps (d), (f), and (g) further comprises utilizing a track to scan by manually moving or electronically controlled automatic moving.

19. The handheld 3D scanning method according to claim 13, wherein the steps (d), (f) and (g) are continuously moving forward along a direction.

20. The handheld 3D scanning method according to claim 13, wherein the step (b) further comprises obtaining the plurality of 3D measuring information of the object to be measured sequentially at different time points by the at least one first 3D sensing module and the at least one second 3D sensing module.

21. The handheld 3D scanning method according to claim 13, wherein in the step (b), each projecting unit of the first 3D sensing modules and the second 3D sensing modules adopts a projecting light source having different frequencies to project, and the at least one image sensing unit filters the light correspondingly to have the obtained 3D measuring information without being mutually interfered.
